(19) United States
(12) Patent Application Publication
(10) Pub. No.: US 2025/0302566 A1
(43) Pub. Date: Oct. 2, 2025

(54) MANUAL RELEASE TOOL

(71) Applicant: MOMENTIS SURGICAL LTD., Or Yehuda (IL)

(72) Inventors: Michael NIKOLAEVSKY, Rishon Lezion (IL); Idan ROTEM, Tel Aviv (IL)

(21) Appl. No.: 18/866,096

(22) PCT Filed: May 10, 2023
(86) PCT No.: PCT/IL2023/050481

§ 371 Date: Nov. 15, 2024

Related U.S. Application Data
(60) Provisional application No. 63/345,108, filed on May 24, 2022.

(51) Int. Cl. A61B 34/00; A61B 34/37

(52) U.S. Cl. CPC A61B 34/74; A61B 34/37

(57) ABSTRACT

An aspect of some embodiments of the invention relates to a manual release tool configured for manually rotating a gear of a motor unit of a motorized robotic system, comprising a body, a knob rotatable around an axis of the body and located on a proximal side of said body and a distal end of said body being narrower than said knob and comprising at least one first gear protruding axially from said distal end; said at least one first gear configured to operatively interact with at least one second gear of said motor unit of said motorized robotic system; where force is transmitted in a plane of said narrow distal end of said body by rotation of said at least one first gear in said plane.

### RELATED APPLICATION/S

This application claims the benefit of priority of U.S. Provisional Patent Application No. 63/345,108 filed on 24 May 2022, the contents of which are incorporated herein by reference in their entirety.

### FIELD AND BACKGROUND OF THE INVENTION

The present invention, in some embodiments thereof, relates to a manual release tool and, more particularly, but not exclusively, to a manual release tool for a surgical mechanical arm of a robotic surgical system.

### SUMMARY OF THE INVENTION

Following is a non-exclusive list including some examples of embodiments of the invention. The invention also includes embodiments which include fewer than all the features in an example and embodiments using features from multiple examples, also if not expressly listed below.

Example 1. A manual release tool configured for manually rotating a gear of a motor unit of a motorized robotic system, comprising:
a. a body;
b. a knob rotatable around an axis of the body and located on a proximal side of said body;
c. a distal end of said body being narrower than said knob and comprising at least one first gear protruding axially from said distal end; said at least one first gear configured to operatively interact with at least one second gear of said motor unit of said motorized robotic system;
wherein force is transmitted by said knob to said at least one first gear using an axially oriented rod; said force being converted into rotation of said at least one first gear perpendicular to a longitudinal axis of said body and conveyed in a plane of said narrow distal end of said body by rotation of said at least one first gear in said plane.

Example 2. The manual release tool according to example 1, wherein said conveying is performed by a series of side by side gears operatively connected to said at least one first gear.

Example 3. The manual release tool according to example 1, further comprising at least one holder configured to hold said manual release tool in place on said robotic mechanism while said at least one first gear is being actuated.

Example 4. The manual release tool according to example 1, further comprising at least one marker on said tool for assisting a user to position said manual release tool on said motor unit.

Example 5. The manual release tool according to example 1, further comprising a motor in said body configured to allow motorized actuation of said at least one first gear.

Example 6. The manual release tool according to example 1, further comprising a cover-opening tool positioned at a distal end of said body and configured to assist a user to open a cover on said motor unit.

Example 7. The manual release tool according to example 1, wherein a width of said knob is from about 10 mm to about 60 mm.

Example 8. The manual release tool according to example 1, wherein a width of said distal end is from about 4 mm to about 10 mm.

Example 9. The manual release tool according to example 1, wherein said distal end is sized and shaped to be inserted in a narrow slot in said motor unit of said motorized robotic system.

Example 10. The manual release tool according to example 9, wherein a width of said slot is from about 6 mm to about 20 mm.

Example 11. The manual release tool according to example 1, wherein a ration between said at least one first gear and said at least one second gear is from about 1:1 to about 1:2.

Example 12. The manual release tool according to example 1, wherein said at least one first gear or said at least one second gear comprises between 10 and 40 teeth.

Example 13. The manual release tool according to example 1, wherein said manual release tool comprises more than one distal end configured to operatively interact with more than one gears of said motor unit of said motorized robotic system.

Example 14. The manual release tool according to example 1, further comprising a cradle configured for storing said manual release tool.

Example 15. The manual release tool according to example 14, further comprising at least one side protrusion on said body configured to interact with said cradle for holding said manual release tool in said cradle.

Example 16. A robotic system comprising:
a. a motor unit comprising at least one gear;
b. at least one robotic arm operated by said motor unit;
c. an electrically controlled motor which powers said motor unit;
d. a manual release tool selectively and fixedly attachable to said motor unit and configured to engage and rotate said at least one gear.

Example 17. The robotic system according to example 16, wherein said manual release tool configured comprises:
a. a body;
b. a knob rotatable around an axis of the body and located on a proximal side of said body;
c. a distal end of said body being narrower than said knob and comprising at least one first gear protruding axially from said distal end; said at least one first gear configured to operatively interact with at least one second gear of said motor unit of said motorized robotic system; and
wherein force is transmitted by said knob to said at least one first gear using an axially oriented rod; said force being converted into rotation of said at least one first gear perpendicular to a longitudinal axis of said body and conveyed in a plane of said narrow distal end of said body by rotation of said at least one first gear in said plane.

Example 18. The robotic system according to example 17, wherein said conveying is performed by a series of side by side gears operatively connected to said at least one first gear.

Example 19. The robotic system according to example 17, further comprising at least one holder configured to hold said manual release tool in place on said robotic mechanism while said at least one first gear is being actuated.

Example 20. The robotic system according to example 17, further comprising at least one marker on said tool for assisting a user to position said manual release tool on said motor unit.

Example 21. The robotic system according to example 17, further comprising a motor in said body configured to allow motorized actuation of said at least one first gear.

Example 22. The robotic system according to example 17, further comprising a cover-opening tool positioned at a distal end of said body and configured to assist a user to open a cover on said motor unit.

Example 23. The robotic system according to example 17, wherein a width of said knob is from about 10 mm to about 60 mm.

Example 24. The robotic system according to example 17, wherein a width of said distal end is from about 4 mm to about 10 mm.

Example 25. The robotic system according to example 17, wherein said distal end is sized and shaped to be inserted in a narrow slot in said motor unit of said motorized robotic system.

Example 26. The robotic system according to example 25, wherein a width of said slot is from about 6 mm to about 20 mm.

Example 27. The robotic system according to example 17, wherein a ration between said at least one first gear and said at least one second gear is from about 1:1 to about 1:2.

Example 28. The robotic system according to example 17, wherein said at least one first gear or said at least one second gear comprises between 10 and 40 teeth.

Example 29. The robotic system according to example 17, wherein said manual release tool comprises more than one distal end configured to operatively interact with more than one gears of said motor unit of said motorized robotic system.

Example 30. The robotic system according to example 17, further comprising a cradle configured for storing said manual release tool.

Example 31. The robotic system according to example 30, further comprising at least one side protrusion on said body configured to interact with said cradle for holding said manual release tool in said cradle.

Example 32. A manual release tool configured for manually rotating a gear of a motor unit of a motorized robotic system, comprising:
a. a body;
b. a knob rotatable around an axis of the body and located on a proximal side of said body;
c. a distal end of said body being narrower than said knob and comprising at least one first gear protruding axially from said distal end; said at least one first gear configured to operatively interact with at least one second gear of said motor unit of said motorized robotic system;
d. at least one motor located inside said body and operatively connected to said at least one first gear and configured for rotating said at least one first gear;
wherein force is transmitted by said motor to said at least one first gear using an axially oriented rod; said force being converted into rotation of said at least one first gear perpendicular to a longitudinal axis of said body and conveyed in a plane of said narrow distal end of said body by rotation of said at least one first gear in said plane.

Example 33. The manual release tool according to example 32, wherein said knob is configured to allow manual actuation of said at least one first gear when said at least one motor cannot be operated.

Example 34. The manual release tool according to example 32, further comprising circuitry configured to control said at least one motor.

Example 35. The manual release tool according to example 34, wherein said circuitry is operable by a user using one or more of a button, an external device connected via wireless connection and a GUI.

Example 36. The manual release tool according to example 32, wherein said conveying is performed by a series of side by side gears operatively connected to said at least one first gear.

Example 37. The manual release tool according to example 32, further comprising at least one holder configured to hold said manual release tool in place on said robotic mechanism while said at least one first gear is being actuated.

Example 38. The manual release tool according to example 32, further comprising at least one marker on said tool for assisting a user to position said manual release tool on said motor unit.

Example 39. The manual release tool according to example 32, further comprising a cover-opening tool positioned at a distal end of said body and configured to assist a user to open a cover on said motor unit.

Example 40. The manual release tool according to example 32, wherein a width of said knob is from about 10 mm to about 60 mm.

Example 41. The manual release tool according to example 32, wherein a width of said distal end is from about 4 mm to about 10 mm.

Example 42. The manual release tool according to example 32, wherein said distal end is sized and shaped to be inserted in a narrow slot in said motor unit of said motorized robotic system.

Example 43. The manual release tool according to example 42, wherein a width of said slot is from about 6 mm to about 20 mm.

Example 44. The manual release tool according to example 32, wherein a ration between said at least one first gear and said at least one second gear is from about 1:1 to about 1:2.

Example 45. The manual release tool according to example 32, wherein said at least one first gear or said at least one second gear comprises between 10 and 40 teeth.

Example 46. The manual release tool according to example 32, wherein said manual release tool comprises more than one distal end configured to operatively interact with more than one gears of said motor unit of said motorized robotic system.

Example 47. The manual release tool according to example 32, further comprising a cradle configured for storing said manual release tool.

Example 48. The manual release tool according to example 47, further comprising at least one side protrusion on said body configured to interact with said cradle for holding said manual release tool in said cradle.

Example 49. A method of actuating a robotic arm, comprising:
a. driving said robotic arm using an electronically controlled motor via a motor unit including at least one first gear;
b. fixedly and temporarily attaching a manual release tool to said motor unit;
c. rotating said at least one first gear using said manual release tool.

Example 50. The method according to example 49, wherein said rotating comprises rotating at least one second gear located at a distal end of said manual release tool and configured to operatively engage said at least one first gear.

Example 51. The method according to example 49, wherein said rotating comprises manually rotating a knob located at a proximal end of said manual release tool.

Example 52. The method according to example 50, wherein said rotating comprises activating at least one motor located inside a body of said manual release tool and configured for rotating said at least one second gear.

Example 53. The method according to example 49, wherein said fixedly and temporarily attaching comprises attaching a holder located in said manual release tool to a dedicated location in said motor unit.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic representation of an exemplary robotic surgical system comprising a surgical mechanical arm, according to some embodiments of the invention;

FIG. 2a is a schematic representation of an exemplary motor unit and exemplary actuation gears for actuation of a device including surgical arms, according to some embodiments of the invention;

FIG. 2b is schematic representation of an exemplary detailed view of the motor units, according to some embodiments of the invention

FIG. 3a is an image of exemplary motor units with external housing, according to some embodiments of the invention;

FIG. 3b is an image of exemplary motor units with external housing with open covers, according to some embodiments of the invention;

FIG. 4a is a schematic representation of an exemplary single manual release tool, according to some embodiments of the invention;

FIG. 4b is a schematic section view representation of an exemplary single manual release tool, according to some embodiments of the invention;

FIG. 4c is a schematic bottom view representation of an exemplary single manual release tool, according to some embodiments of the invention;

FIG. 5a is a schematic representation of a motor unit with the removable cover open, according to some embodiments of the invention;

FIG. 5b is a schematic representation of a motor unit with the removable cover open and a plurality of manual release tools attached to the motor unit, according to some embodiments of the invention;

FIG. 5c is a section view of exemplary motor units with exemplary manual release tools, according to some embodiments of the invention

FIG. 5d is a schematic view of the surgical arms, according to some embodiments of the invention

FIG. 5e is a schematic view of the surgical arms, according to some embodiments of the invention

FIG. 5f is a cross-section of the motor construct along the length of the construct, showing first motor unit, according to some embodiments of the invention;

FIG. 5g is a cross section of the motor construct along an axis perpendicular to the long axis, according to some embodiments of the invention;

FIG. 6a is a schematic representation of an exemplary flap with dedicated grooves, according to some embodiments of the invention;

FIG. 6b is a schematic representation of dedicated markings in the flap and/or in the manual release tool, according to some embodiments of the invention;

FIG. 7 is a flowchart of an exemplary method according to some embodiments of the invention; and

FIGS. 8a-b are schematic representations of a manual release tool in a dedicated cradle and a dedicated cradle, respectively, according to some embodiments of the invention.

### DESCRIPTION OF SPECIFIC EMBODIMENTS OF THE INVENTION

The present invention, in some embodiments thereof, relates to a manual release tool and, more particularly, but not exclusively, to a manual release tool for a surgical mechanical arm of a robotic surgical system.

### Overview

An aspect of some embodiments of the invention relates to a manual release tool for manually actuating a motor unit of an automatic robotic device. In some embodiments, the tool comprises a handle with a knob at a proximal end and an actuation distal end sized and shaped to be inserted into a narrow space within the motor unit, the narrow space allowing access to at least one gear of the motor unit. In some embodiments, the tool comprises a holder configured to reversibly lock the tool in place on the motor unit while the tool is being actuated. In some embodiments, the locking place is a flap on the motor unit. In some embodiments, the flap can be either open or close while using the tool. In some embodiments, the motor unit comprises a dedicated groove for the holder. In some embodiments, the groove is on the top surface of the flap, on the bottom surface of the flap or on both surfaces of the flap. In some embodiments, the motor unit is configured to allow the interconnection of more than one tool at a time, therefore allowing a user to manually actuate more than one part of the robotic device. In some embodiments, since motor units are required to be compact, there is no much space within the motor units to allow the insertion of external tools, therefore, in some embodiments, the manual release tool is small enough to be inserted in a small dedicated opening in the motor unit, which allows an operational interaction between the manual release tool and mechanisms in the motor unit. In some embodiments, the distal end of the tool comprises at least one gear configured to interact with at least one gear in the motor unit. In some embodiments, actuation of the knob causes the rotation of the gear at the distal end, which rotates the gear in the motor unit. In some embodiments, the ration between the gear of the tool and the gear of the motor unit is from about 1:1 to about 2:1, or for example from about 1:1 to about 1:2. For example, in the case of a ratio of 2:1, two full rotations of the knob will cause one rotation of the gear in the motor unit. It should be understood that different ratios can be used according to the specific requirements of the motor unit, and that the abovementioned example (or the below-mentioned example) is not intended to be limiting in any way. In some embodiments, the manual release tool is kept in a dedicated cradle configured to reversibly hold (storage) the tool while not is use. In some embodiments, the cradle is kept on the robotic surgical system. In some embodiments, the cradle is kept in the vicinity of the robotic surgical system. In some embodiments, a robotic surgical system comprises one or more manual release tools, for example, a robotic surgical system having two motor units may require six independent manual release tools to actuate six distinct gears in the motor unit. In some embodiments, the manual release tool is a multi-manual release tool comprising more than one distal end, for example two or three distal ends, each comprising a gear configured to interact with a specific gear in the motor unit. In some embodiments, the multi-manual release tool comprises a selector (for example a switch) configured to choose which, either one or more than one, distal end to actuate. In some embodiments, the manual release tool is not manual, but rather motorized release tool. In some embodiments, the motorized release tool comprises a motor within the body of the tool configured to actuate the gear at the distal end of the tool. In some embodiments, the motorized release tool comprises a dedicated button configured to actuate the motor of the tool.

An aspect of some embodiments of the invention relates to a manual release tool configured to be inserted in a small space in a robotic surgical system. In some embodiments, a potential advantage of the manual release tool is that it potentially overcomes the difficulties of designing and generating a device that can be inserted in such small spaces within the complicated and intricate mechanisms of the robotic surgical system, especially, since the gears that are required to be manually actuated are positioned far inside this small space and, possibly, in close proximity to delicate electronics, which, in addition, increases the potential danger of damaging the delicate electronics while using manual tools in general. Therefore, in some embodiments, the manual release tool of the present invention comprises a long and narrow distal end configured to reach the mechanical gear located far inside the robotic surgical system and actuate it while in parallel avoiding making contact with the delicate electronics of the robotic surgical system.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

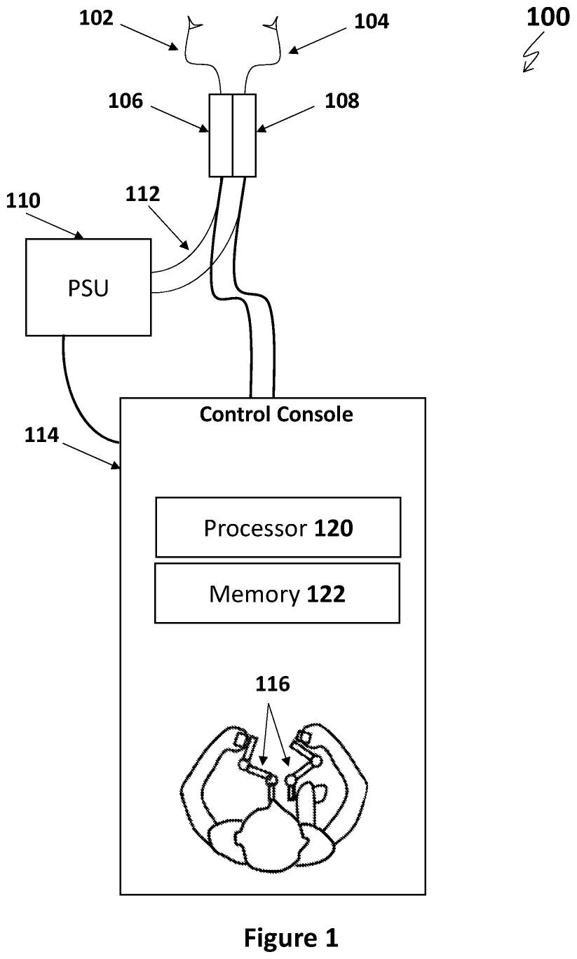

Referring now to FIG. 1 showing a schematic representation of an exemplary robotic surgical system comprising a surgical mechanical arm, according to some embodiments of the invention. In some embodiments, an exemplary robotic surgical system 100 comprises at least one surgical mechanical arm, for example, a plurality of surgical mechanical arms 102, 104. In some embodiments, surgical mechanical arms are sized and/or shaped for insertion into a human body (not shown). In some embodiments, the system 100 comprises at least one motor unit, for example, a plurality of motor units 106, 108, where each of surgical mechanical arms 102, 104 is actuated by a robotic control unit (RCU) using one or more motor unit. For example, where a first surgical arm 102 is actuated by a first motor unit 106 and/or a second arm 104 is actuated by a second motor unit 108. In some embodiments, the surgical mechanical arm 102/104 are supplied with power by the motor unit 106/108 to which it is attached. In some embodiments, surgical arms are supplied with power by an electrosurgical power supply generator 110. In some embodiments, supply to the motor units 106/108 is via cable/s 112. In some embodiments, the surgical arms 102/104 are controlled at a control console 114. In some embodiments, the control console 114 is configured to allow manual control of the surgical mechanical arms 102, 104 and or to allow automatic control of the surgical mechanical arms 102, 104 by means of a dedicated computer and/or software (see below). In some embodiments, the control console 114 comprises one or more user interfaces: for example, in some embodiments, control console 114 comprises one or more input device arm 116 where the control console is configured to generate control signals upon movement of the arm/s (e.g. in some embodiments, the arms generate control signals when moved). In some embodiments, one or more input device arm 116 includes an additional user interface (not illustrated), for example, one or more button and/or switch. In some embodiments, control console 114 comprises a processor 120 configured to receive signals from user input/s and to send control signals to motor units 106/108 and/or electrosurgical power supply unit 110. In some embodiments, the processor 120 receives an indication of a selection of an electrosurgical operational mode, for example, for each of the surgical arms 102, 104, and/or motor units 106, 108.

### Exemplary Motor Unit

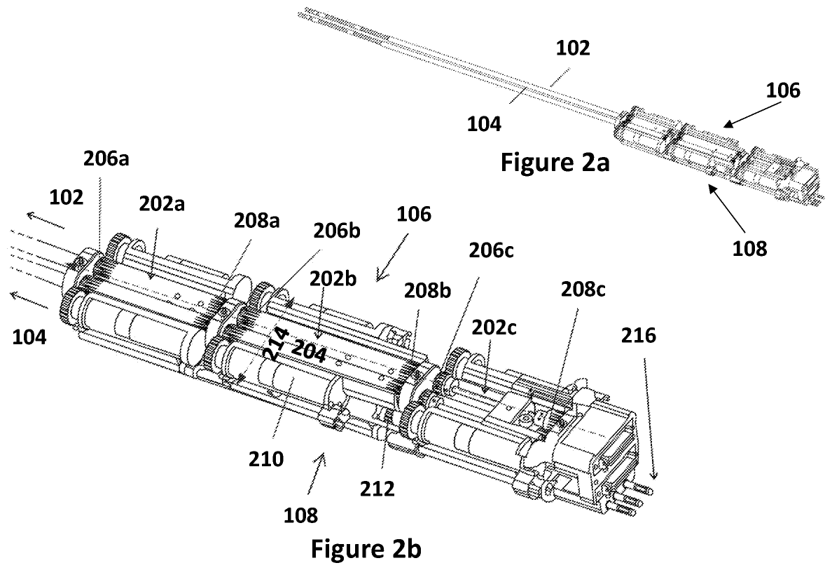

In some embodiments, the motor unit (106/108) actuate the surgical arms (102/104) to perform one or more movements. In some embodiments, in an initial state at the beginning of the use of the robotic surgical system 100, the surgical arms (102/104) are positioned in the straight position. In some embodiments, this initial straight position is used for the insertion of the surgical arms (102/104) into the patient. In some embodiments, once the surgical arms (102/104) are inside the body of the patient, the motor units (106/108) are activated to actuate the surgical arms (102/104) and cause them to bend in one or more locations along the surgical arms (102/104) and to rotate in order to allow the user to reach the desired location inside the body of the patient. In some embodiments, the motor units (106/108) comprise a plurality of gears configured to actuate the different bending locations in the surgical arms (102/104). Referring now to FIG. 2a, showing a schematic representation of an exemplary motor unit and exemplary actuation gears for actuation of a device including surgical arms, according to some embodiments of the invention. As referred to in FIGS. 2a-2b, an exemplary robotic surgical system 100 comprises two motor units (106/108), each coupled to an arm (102/104) and configured for actuating movement of the arm. FIG. 2b is a schematic representation of an exemplary detailed view of the motor units (106/108), according to some embodiments of the invention. In some embodiments, a first motor unit 106 configured for actuating arm 102 comprises, for example, 3 actuation mechanisms 202a, 202b and 202c. In some embodiments, similarly, second surgical arm 104 is actuated by a motor unit 108 comprising three actuation mechanisms. Optionally, the motor units are parallel to each other. Optionally, the motor units are arranged such that the actuation mechanisms are symmetrically arranged along a long axis 204 of the motor units. In some embodiments, a first actuation mechanism 202a, including first rotation gear 206a and first bending gear 208a, drives flexion/extension and rotation of a shoulder joint in the arm. Similarly, in some embodiments, a second actuation mechanism 202b, including second rotation gear 206b and second bending gear 208b, drives flexion/extension and rotation of an elbow joint in the arm. In some embodiments, one or more driving gear coupled to a motor 210 is disposed underneath the motor unit 108. For example, in some embodiments, a gear which drives second bending gear 208b, which gear is coupled to a motor is disposed on an underside of the motor unit 106. For example, gear 212 drives a second actuation mechanism corresponding to second surgical arm 104. Similarly, in some embodiments, a third actuation mechanism 202c, including third rotation gear 206c and third bending gear 208c, actuates an end effecter (e.g. opens and closes a gripper) and drives rotation of a wrist joint in the arm.

In some embodiments, a motor unit is small. In some embodiments, a motor unit comprises a long axis length 204 of between 100-600 mm, or 200-400 mm, or 300-500 mm, or 150-400 mm, or intermediate, longer or shorter length.

In some embodiments, for example as shown herein, a motor construct comprising two parallel arrangements for actuating two arms comprises a width 214 (e.g. as measured perpendicular to the long axis) between 20-100 mm, or 30-80 mm, or 50-70 mm, or intermediate, longer or shorter size.

In some embodiments, motor 210 is cylindrical. Optionally, a diameter of motor 210 is less than 17 mm, less than 35 mm, less than 10 mm or intermediate, larger or smaller diameters. A potential advantage of disposing a motor of a relatively small diameter in a parallel position relative to the arm may include maintaining the dimensions of the motor unit small.

Alternatively, the motor is not cylindrical, for example rectangular. In some embodiments, the motor comprises a hollow shaft. A potential advantage of a hollow shaft may include reducing the footprint of the system in the operating room.

In some embodiments, electrical power is supplied through wires to the motor units, for example, in some embodiments, contacts 216 are connected to an electrical power supply. The electrical power supply may include a battery (optionally rechargeable) and/or a generator and/or connection to the electrical network via a wall socket and/or a combination thereof. In some embodiments, the power range is between 100-300 W, for example 150W, 200 W, 250 W or intermediate, higher or lower ranges. In some embodiments, an uninterruptible power supply source is used to protect from power interruptions.

In some embodiments, a motor construct drives more than two surgical arms and/or drives additional device elements. For example, in some embodiments, a motor construct drives two device arms and a camera.

In some embodiments, the motor unit comprises a motherboard, optionally underlying the actuation mechanisms. In some embodiments, one or more driver circuits are operably coupled to motherboard for controlling operation of the motor unit. In some embodiments, each driver circuit is configured to control activation of one of the motors.

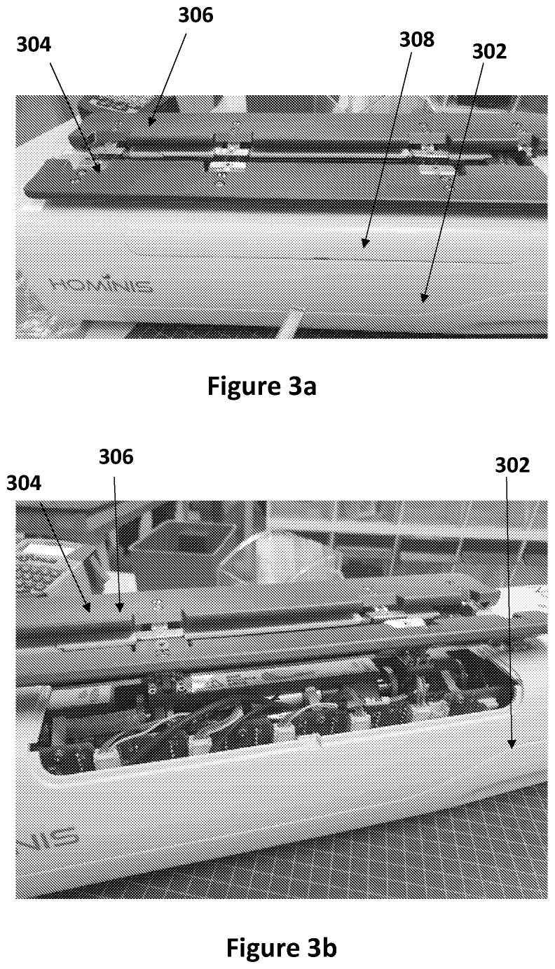

Referring now to FIG. 3a and FIG. 3b, showing an image of exemplary motor units with external housing, according to some embodiments of the invention. In some embodiments, the motor units comprise an external housing 302 configured to enclose the plurality of motors, gears and electronic components of the motor units 106/108. In some embodiments, additionally, the surgical arm is mechanically held in position by one or more component. In some embodiments, motor unit 106/108 includes one or more clamping hammer which contact and/or apply pressure to the surgical arm. In some embodiments, clamping hammers are brought into contact and apply pressure to surgical arm when a flap 304/306 is rotated about a hinge attachment to motor unit to a closed position illustrated in FIG. 3a. In some embodiments, additionally, the external housing 302 comprises a removable cover 308 configured to allow access to the plurality of motors, gears and electronic components of the motor units 106/108, as shown for example in FIG. 3b.

In some embodiments, if and when there is a problem with one of motors/gears/electronics in one or more of the motor units, and/or in general, a disconnection between the control units and the arms, an electrical problem, a software problem and/or a mechanical problem, the system is configured to allow a user to manually actuate the gears of the motor units to allow bringing the arms to an initial straight position, and also to release a grabbed tissue by opening the gripper (when necessary) and therefore allow the retraction of the arms 102/104 from the inside of the patient.

In some embodiments, manual actuation of the gears is performed using one or more of manual release tools, as will be further disclosed below.

### Exemplary Single Manual Release Tool

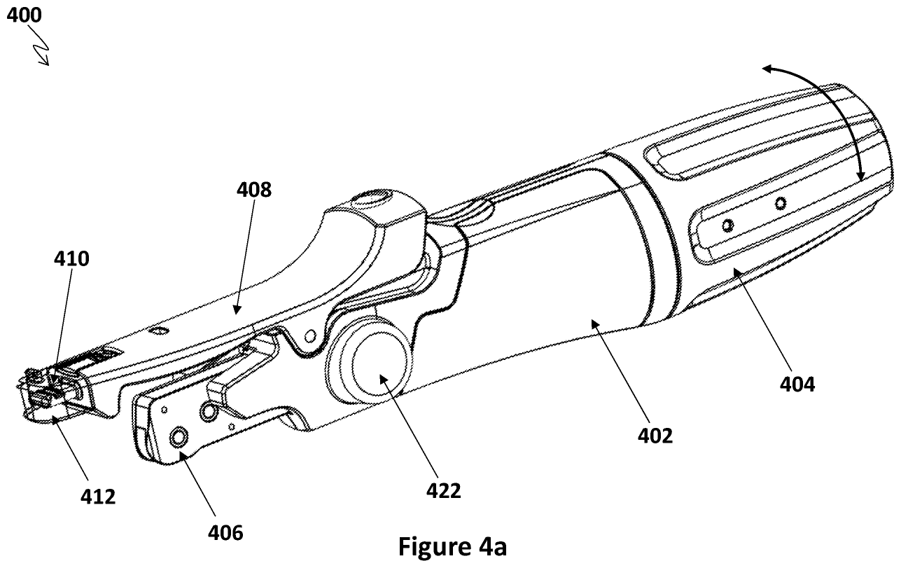

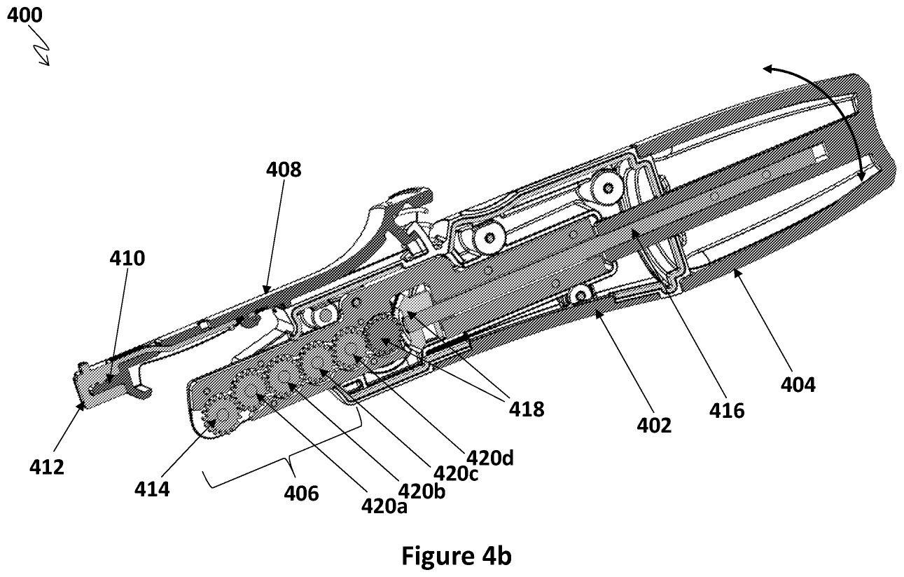

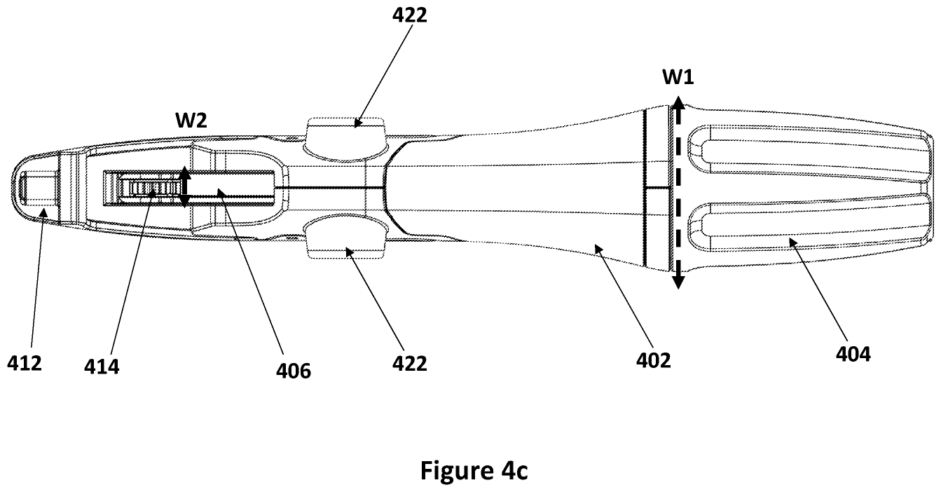

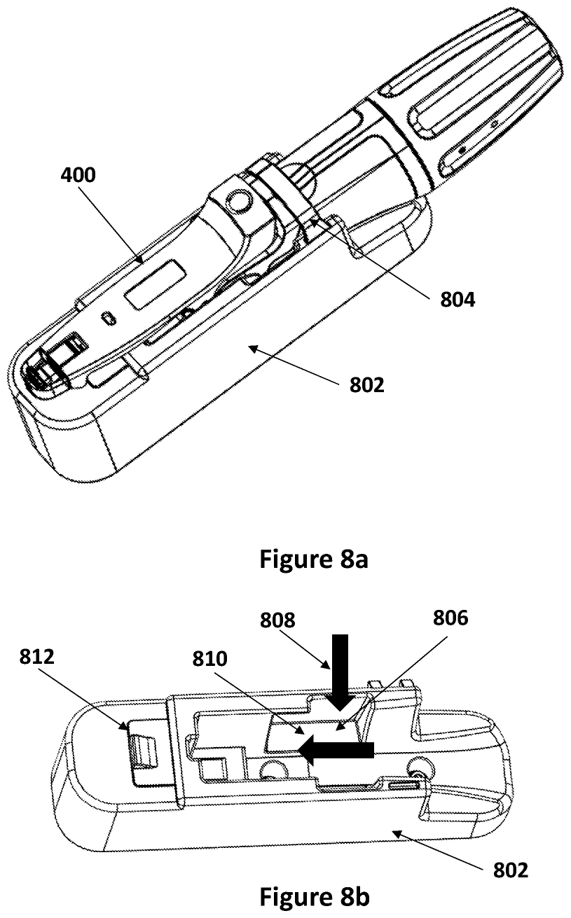

Referring now to FIG. 4a, showing a schematic representation of an exemplary single manual release tool 400, according to some embodiments of the invention. In some embodiments, the single manual release tool 400 comprises a body 402 comprising a knob 404 at a proximal end of the body 402. In some embodiments, the single manual release tool 400 comprises a distal end 406 comprising at least one gear (not shown in FIG. 4a—see below) configured to interconnect with at least one gear of the motor unit, as will be further explained below. In some embodiments, the single manual release tool 400 optionally comprises a holder 408 configured to hold in place the single manual release tool 400 to the motor unit while its being actuated by a user. In some embodiments, the single manual release tool 400 optionally comprises a cover opener 410, optionally enclosed by a removable cap 412, and configured to assist in opening the removable cover in the motor unit. In some embodiments, the single manual release tool 400 comprises one or more side protrusions 422 configured to assist in holding the single manual release tool 400 inside a dedicated cradle (802 in FIG. 8a and FIG. 8b) used for storage of the single manual release tool 400 while not in use. For example, in FIG. 4a, the protrusions 422 are two rounded bodies extending from the body 402 of the single manual release tool 400 and adapted to enter the channels 806 in the cradle 802 (see FIG. 8b).

Referring now to FIG. 4b, showing a schematic section view representation of an exemplary single manual release tool 400, according to some embodiments of the invention. In some embodiments, as explained for FIG. 4a, the single manual release tool 400 comprises a body 402 comprising a knob 404 at a proximal end of the body 402. In some embodiments, the single manual release tool 400 comprises a distal end 406 comprising at least one gear 414 (in FIG. 4b are shown a series of gears) configured to interconnect with at least one gear of the motor unit, as will be further explained below. In some embodiments, the single manual release tool 400 optionally comprises a holder 408 configured to hold in place the single manual release tool 400 to the flap of the motor unit while its being actuated by a user. In some embodiments, the single manual release tool 400 optionally comprises a cover opener 410, optionally enclosed by a removable cap 412, and configured to assist in opening the removable cover of the motor unit. In some embodiments, the knob 404 comprises a rod 416 located along its axis, which rotates when the knob 404 is rotated. In some embodiments, at the distal end of the rod 416 there is a bevel gear 418 configured to convert the rotational force and torque delivered by the rod from the axis of rotation of the rod to a direction which is perpendicular to the axis of rotation, therefore actuating the at least one gear 414, or the plurality of gears 414 and 420a-d in the distal end 406 of the single manual release tool 400. In some embodiments, the knob 404 is configured to rotate to either direction, each direction actuates the gear 414 to a specific direction.

Referring now to FIG. 4c, showing a bottom view representation of an exemplary single manual release tool 400, according to some embodiments of the invention. In some embodiments, the proximal end of the single manual release tool 400 is bigger than the distal end 406, or rather the distal end 406 of the single manual release tool 400 is thinner than the proximal end 404. In some embodiments, W1>W2, as shown for example in FIG. 4c. In some embodiments, the width of the proximal end where the knob 404 is located is from about 30 mm to about 40 mm, optionally from about 20 mm to about 50 mm, optionally form about 10 mm to about 60 mm, for example 35 mm, 38 mm, 40 mm, just to name a few. In some embodiments, the width of the distal end 406 is from about 6 mm to about 8 mm, optionally from about 5 mm to about 9 mm, optionally from about 4 mm to about 10 mm, for example 7 mm, 8 mm, 5 mm, just to name a few. In some embodiments, the proximal end is sized and shaped be ergonomic and potentially allow a user to easily and comfortably hold and actuate the single manual release tool 400. In some embodiments, the distal end 406 of the single manual release tool 400 is sized and shaped to access a dedicated slot in the motor unit, which allows access of the single manual release tool 400 to at least one gear in the motor unit.

In some embodiments, for example, the gear 414 of the manual release tool comprises 18 teeth and the gear 514 of the motor unit comprises 22 teeth, therefor there will be a ratio of about 1:1.2, which means that a full turn of the knob will turn the gear 514 almost a full turn. In some embodiments, for example, in order to flex the elbow (see below) or the shoulder (see below) of the surgical arm (102/104) from a completely bended configuration to a completely straight configuration, three full turns of the knob are required. In some embodiments, four full turns of the knob are required to completely open the hand tool from a completely closed configuration. It should be understood that these numbers are provided just an example to allow a person having skills in the art to understand the invention. In some embodiments, the number of teeth in either gear 414 of the manual release tool or the gear 514 of the motor unit can be different, for example between 15 teeth and 30 teeth. Optionally between 10 teeth and 40 teeth. In some embodiments, the ration between the gears is from about 1:1 to about 1:2.

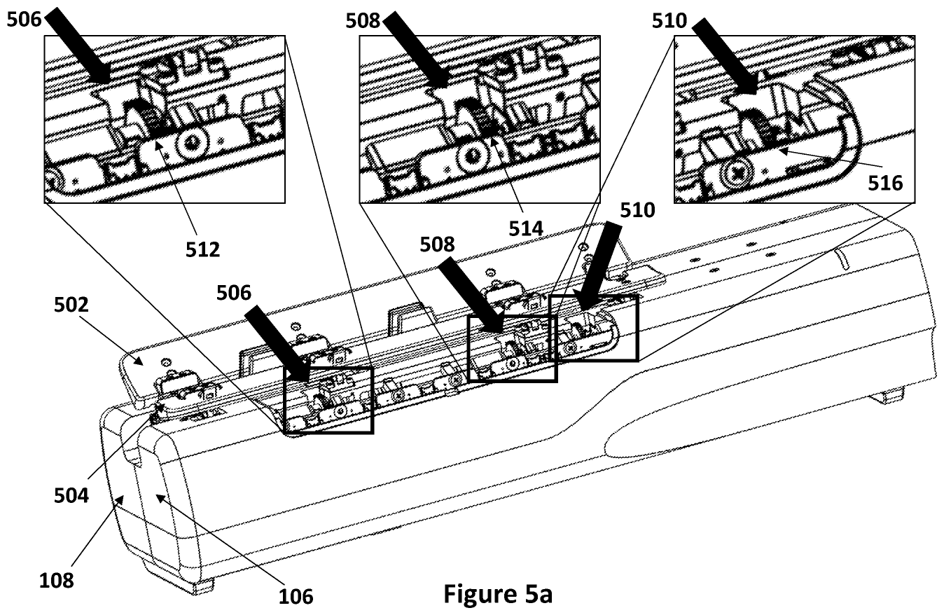

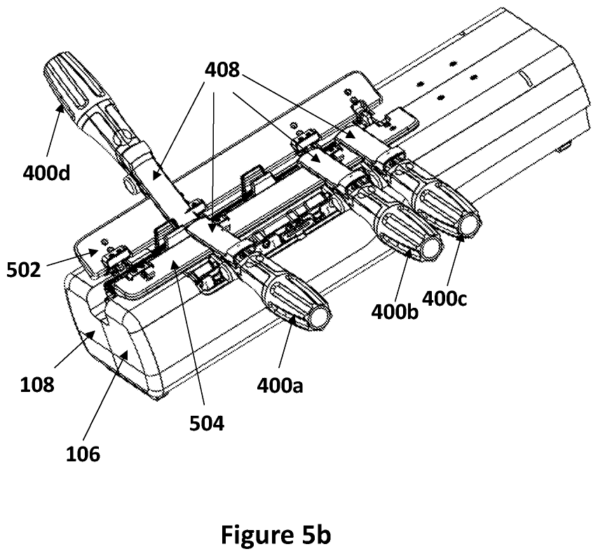

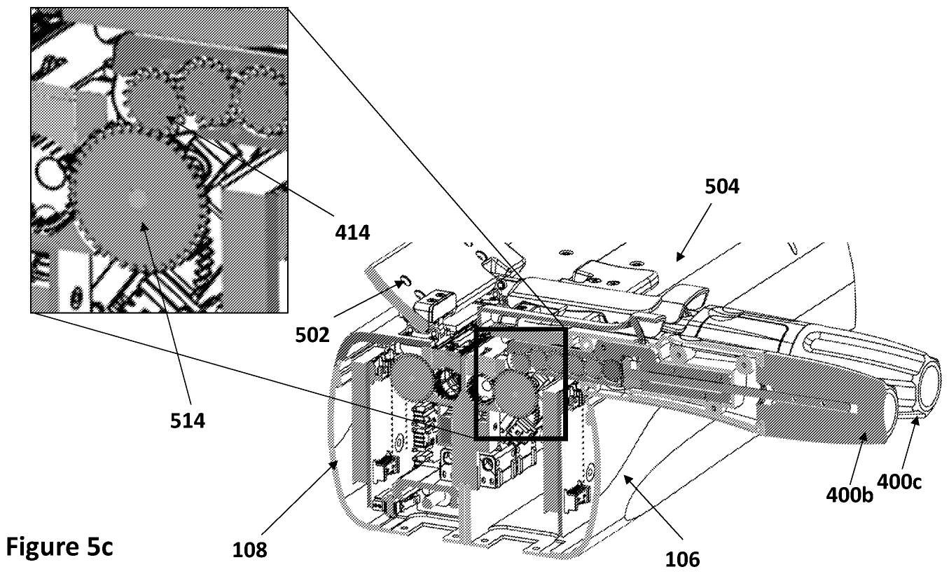

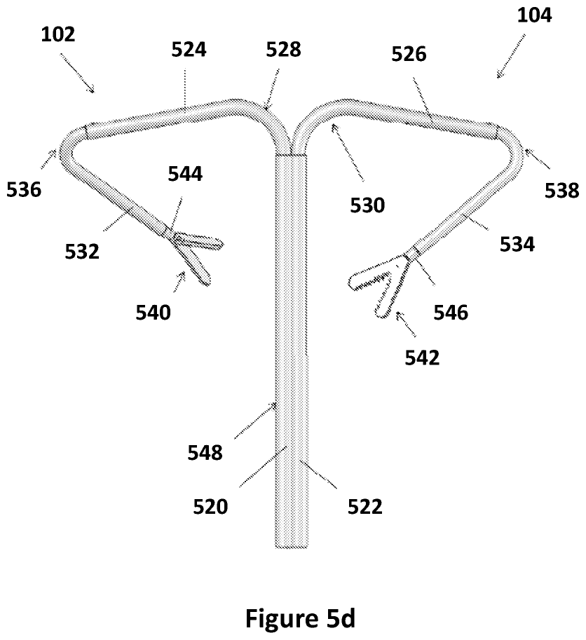

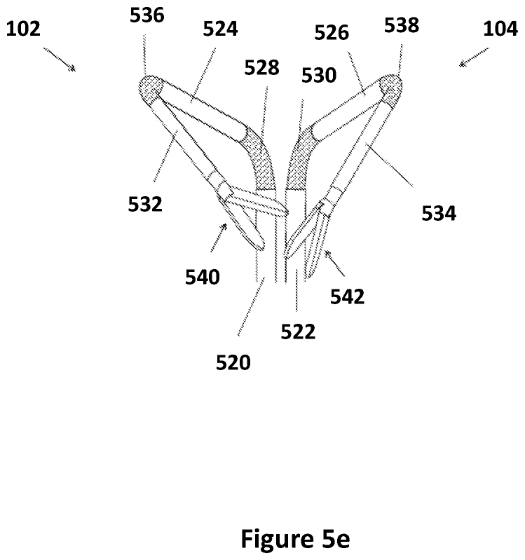

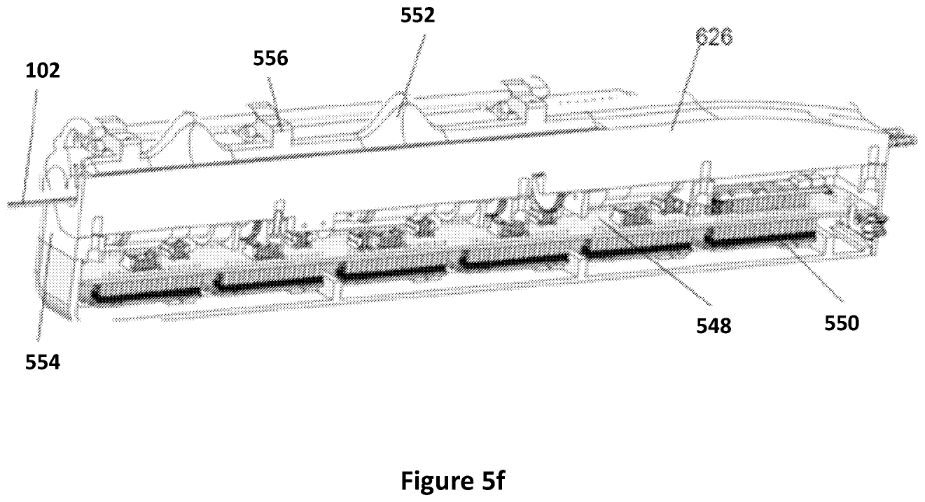

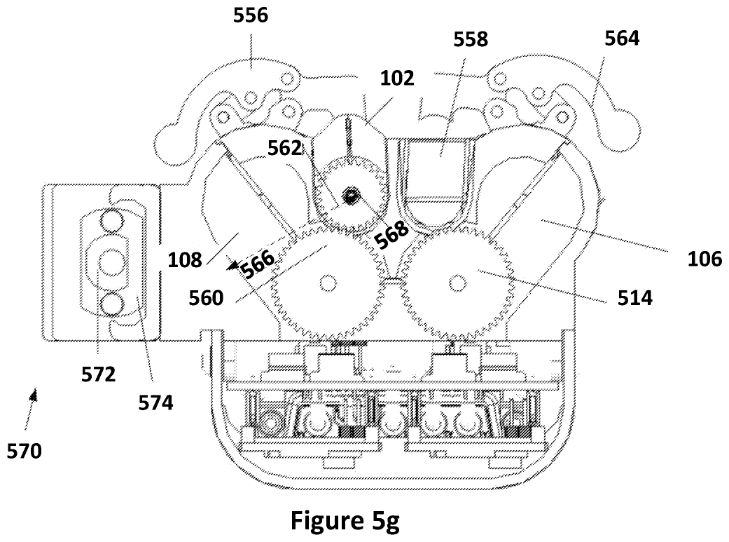

Referring now to FIG. 5a, showing a schematic representation of a motor unit with the removable cover open, according to some embodiments of the invention. In some embodiments, as mentioned above, the motor unit 106/108 comprise one or more dedicated slots 506/508/510 configured to allow access of the single manual release tool 400 to at least one gear 512/514/516 in the motor unit, as schematically shown, for example, in the zoom-in pictures of the slots in motor unit 104 in FIG. 5a. In some embodiments, the distal end 406 of the single manual release tool 400 is sized in shaped to fit within the dedicated slot and reach the at least one gear in the motor unit. In some embodiments, the one or more dedicated slots 506/508/510 are from about 8 mm to about 16 mm, optionally from about 6 mm to about 20 mm, optionally from about 5 mm to about 25 mm, for example 12 mm, 14 mm, 18 mm, just to name a few.

In some embodiments, surgical arm (102/104) is mechanically held in position by one or more components (see below for more details of the exemplary mechanism that actuates the surgical arms). In some embodiments, motor unit (106/108) includes one or more clamping hammer which contact and/or apply pressure to the surgical arm. In some embodiments, clamping hammers are brought into contact and apply pressure to surgical arm (102/104) when a flap 502/504 is rotated about a hinge attachment to motor unit (106/108) to a closed position as shown for example in FIG. 5a. In some embodiments, the holder 408 of the single manual release tool 400 is configured to be reversibly attached to the flap 502/204 to allow holding the single manual release tool 400 in place while it is being actuated by the user, as shown for example in FIG. 5b. FIG. 5b shows four exemplary single manual release tools 400a-d attached to the motor units, three tools attached to flap 504 on motor unit 106 and one tool attached to flap 502 on motor unit 108. In some embodiments, the holder 408 is configured to be actuated (pressed) in order to release the tool from the flap.

In some embodiments, the manual release tool is configured to be used when the flap is open and also when the flap is closed. In some embodiments, the manual release tool optionally comprises an adaptor (not shown) configured to allow anchoring of the tool when the flap is open. In some embodiments, an adaptor is not needed. In some embodiments, no anchoring is required for the use of the manual release tool.

Referring now to FIG. 5c, showing a section view of exemplary motor units with exemplary manual release tools, according to some embodiments of the invention. FIG. 5c shown a section view showing both motor units 102/104 and a cross section of exemplary manual release tool 400b attached to flap 504 in motor unit 106, and exemplary manual release tool 400c attached next to it on the same flap/motor unit. In the zoom-in picture can be seen the interconnection between the at least one gear 414 of the manual release tool 400b and the at least one gear 514 of the motor unit 106. In some embodiments, the interconnection is a fit connection which allows the transmission of forces from the tool to the gear in the motor unit, which will actuate the arms of the robotic surgical system 100.

In order to allow a person having skills in the art to understand the invention, the following paragraphs will explain the connection between the surgical arms (102/104) and the motor units (106/108) and the movements performed by the surgical arms when actuated by the motor units under the command of the robotic control unit (RCU). Referring now to FIG. 5d and FIG. 5e, showing simplified schematic views of the surgical arms (102/104), according to some embodiments of the invention. In some embodiments, as mentioned above, the surgical device includes a first arm 102 and a second arm 104. In some embodiments, one or both surgical arms are sized and/or shaped for insertion into a human body.

In some embodiments each arm (102/104) includes a support segment (520/522), coupled to a first segment (524/526) by a first connecting section (528/530), where first segment (524/526) is coupled to a second segment (532/534) by a second connecting section (536/538), and a third segment (540/542) coupled to second segment (532/534) by a third connecting section (544/546). In some embodiments, one or more of support segments (520/522) are rigid. In some embodiments one or more of support segments (520/522) are flexible or include a flexible portion. In some embodiments, support segments (520/522) are coupled, e.g. by a cover 548. In some embodiments, support segments (520/522) are coupled at only a portion of the torso length or are not coupled. In some embodiments, one or more arm includes a humanoid like structure. For clarity, in some portions of this document, device segments and connecting sections are referred to by anatomical names: Support segments (520/522) are also termed first torso 520 and second torso 522, first connecting sections (528/530) are also termed first shoulder joint 528, second shoulder joint 530, first segments (524/526) are also termed first humerus 524 and second humerus 526, second connecting sections (536/538) are also termed first elbow joint 536, and second elbow joint 538, second segments (532/534) are also termed first radius 532 and second radius 534 and third segments (540/542) are also termed first hand tool 540 and second hand tool 542.

In some embodiments, one or more connecting section includes a hinge. In some embodiments, one or more connecting section is flexible and/or includes a flexible portion. In an exemplary embodiment, a device arm includes an elbow joint and a shoulder joint where bending of the joint is distributed along the joint in a direction of a joint long axis.

In some embodiments, torsos (520/522) are close together, for example, a long axis of first torso 520 and a long axis of second torso 522 are within 5 mm, or 3 mm, or 1 mm of each other. Alternatively, torsos (520/522) are spaced apart from each other. Additionally or alternatively, torsos (520/522) are configured to converge or to diverge relative to each other. Optionally, a torso is curved.

In some embodiments, one or more device segment has a substantially cylindrical external shape (e.g. radius, humerus). In some embodiments, joints have circular long axis cross-section. Alternatively, in some embodiments, one or more device segment and/or joint has non-circular cross section external shape, for example, oval, square, rectangular, irregular shapes. In some embodiments, a surgical arm includes one or more short and/or adjustable segment. In some embodiments, flexible portions are directly connected. In some embodiments, a flexible portion comprises a plurality of stacked links.

In some embodiments, for example, so that rotation of a joint also causes rotation of joints distal of the rotated joint, more than one actuation mechanism is driven in rotation of the joint. For example, in some embodiments, for rotation of the shoulder joint, gears 206a, 208a, 206b, 208b, 206c, 208c (FIG. 2b) are rotated in the same direction. For example, in some embodiments, for rotation of the elbow joint, gears 206b, 208b, 206c, 208c (FIG. 2b) are rotated in the same direction. For example, in some embodiments, for rotation of the end effecter, gears 206c, 208c (FIG. 2b) are rotated in the same direction. In some embodiments, concurrent rotation of nested portions with outer portions prevents stress on and/or tangling of internal elongated elements (e.g. elongated element/s which are used to effect flexion/extension, e.g. elongated element/s providing power supply).

In some embodiments, one or more actuation mechanism is used to flex/extend a joint. For example, in some embodiments, to bend a shoulder joint, elongated elements for bending of both the shoulder joint and elbow joint are moved, for example by actuating bending gear 208a and bending gear 208b (FIG. 2b).

In some embodiments, if elongated elements for the elbow are not moved and/or released, tension in the elongated elements associated with the elbow joint resist movement of the shoulder joint.

In some embodiments, a motor construct drives more than two surgical arms and/or drives additional device elements. For example, in some embodiments, a motor construct drives two device arms and a camera.

FIG. 5f is a cross-section of the motor construct along the length of the construct, showing first motor unit 106, according to some embodiments.

In some embodiments, as mentioned above, the motor unit comprises a motherboard 548, optionally underlying the actuation mechanisms. In some embodiments, one or more driver circuits 550 are operably coupled to motherboard 548 for controlling operation of the motor unit. In some embodiments, each driver circuit is configured to control activation of one of the motors (e.g. one of the 6 motors described hereinabove). In some embodiments, cross-control of the motors is provided. In an example, a position sensor of a first motor is controlled by a controller of a second motor. Optionally, in such configuration, malfunctioning of the first motor can be detected by the controller of the second motor. In some embodiments, malfunction of the first motor is detected by the controller of the second motor.

In some embodiments, an external housing 302 of the motor unit comprises a handle 552 for attaching and/or releasing arm 102 from a distal end face 554 of the motor unit.

In some embodiments, one or more latches 556 are configured on external housing. Optionally, latch 556 is configured to release a gear fixation mechanism used, for example, during attachment of the surgical arm to the motor unit to maintain calibration of the motor unit, for example as further described herein.

FIG. 5g is a cross section of the motor construct along an axis perpendicular to the long axis, according to some embodiments. In some embodiments, the motor construct is configured to actuate two surgical arms; in this example, one surgical arm 102 (an extension of which) is shown to be received within the first motor unit 106, while the second opposing motor unit 108 is shown in a configuration suitable for receiving a second arm, for example received within internal lumen 558.

It is noted that in some embodiments a motor unit configured for actuating a single arm is comprised of only of one of the sides of the motor construct shown herein, including, for example, 3 actuation mechanisms.

In some embodiments, for example as shown herein, actuation gears 514 and 560 of motors 106 and 108 respectively are each configured to drive a gear of an actuation mechanism, for example actuation gear 514 of motor 106 is configured to drive rotation gear or bending gear 562 (such as gear 206a or 208a or 206b or 208b or 206c or 208c).

In some embodiments, latch 556 configured at motor unit 108 which, in this illustration, includes the arm, is shown at a closed position. In some embodiments, a closed positioned of the latch releases a fixation mechanism of gear 562, allowing it to rotate freely. As further shown in FIG. 5g, a second latch 564 configured at the second motor unit 106 is shown at an open, lifted position.

In some embodiments, a motor such as 108 is disposed such that it does not extend to a distance 566 longer than 5 mm, 10 mm, 20 mm or intermediate, longer or shorter distances relative to a central long axis of an actuation mechanism, for example passing through a center 568 of rotation/bending gear. A potential advantage of a motor disposed adjacent an actuation mechanism, optionally in parallel to the actuation mechanism such that it substantially does not protrude outwardly or protrudes outwardly to a short distance only may include reducing bulkiness of the motor unit, potentially allowing insertion of the surgical arm(s) as well as the motor unit into the body during operation.

In some embodiments, the motor unit is coupled to a linear unit 570, configured for actuating linear movement of the motor unit (and thereby of the arm(s)), for example actuate advancement and/or retraction of the device to and/or from the patient body. In some embodiments, linear unit 570 comprises a rail 572 on which a sliding element 574 coupled to the motor unit can be moved linearly. In some embodiments, movement (e.g. sliding) of the motor unit on the rail of the linear unit is actuated by a motor.

Alternatively, in some embodiments, the linear unit is an integral component of the motor unit.

In some embodiments, the linear unit comprises one or more sensors, such as microswitches, for detecting movement of the motor unit. In some embodiments, the linear unit comprises one or more actuation buttons configured to provide for a user (e.g. nurse) to move the motor unit according to the need. In some embodiments, the motor driving the linear movement (not shown herein) comprises an electro-magnetic brake. Optionally, the brake is configured to avoid unwanted movement (e.g. slipping) of the motor unit, for example during a power outage.

### Exemplary Assisting Means for Placing Tool in Place

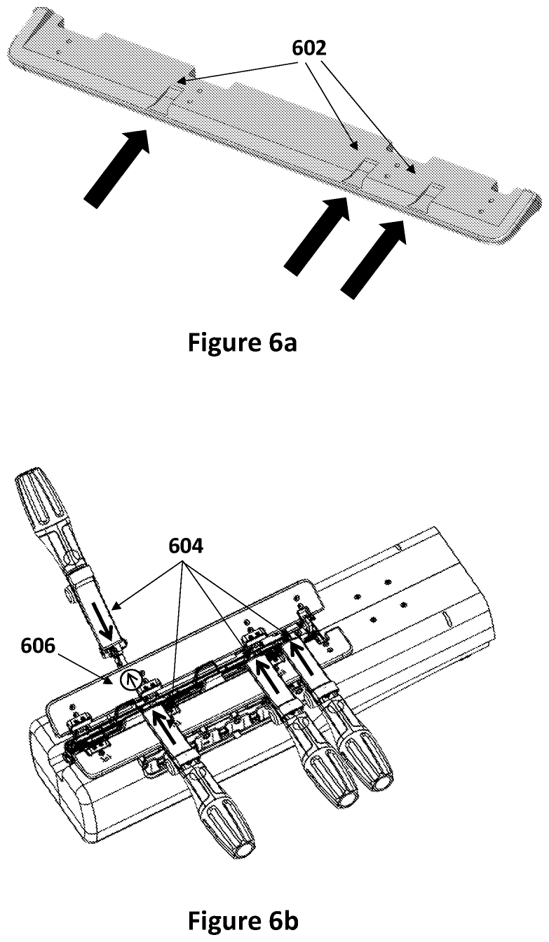

In some embodiments, the robotic surgical system 100 comprises assisting means to facilitate the insertion of the manual release tool into place in the motor unit. For example, as shown in FIG. 5a, the motor unit comprises actual grooves that can be visually identified by the user, which assist the user in identifying the location of insertion of the tool in the motor unit. In some embodiments, as shown for example in FIG. 6a, optionally, the flap 502/504 comprises dedicated grooves 602 on the surface, which indicate the locations where the user needs to insert the manual release tool. In some embodiments, the grooves are on the top surface of the flap. In some embodiments, the groves are on the bottom surface of the flat. In some embodiments, the groves are both on the top surface and on the bottom surface of the flap. Additionally or alternatively, in some embodiments, the tool is marked 604 and the flap is marked 606 so as to show the user the location of the insertion of the tool in the motor unit, as shown for example in FIG. 6b.

### Exemplary Multiple Manual Release Tool

In some embodiments, instead of having individual single manual release tools, each connecting to a different position in either location on the motor units, there is a multiple manual release tool having one proximal end, configured to be held and actuated by a user and having, for example, three distal ends separated at predetermined distances, which match the three locations on the motor unit where the release tools are positioned. In some embodiments, when the knob is actuated, either one, or two or all three distal ends are actuated, according to the choice of the user. In some embodiments, the proximal end comprises individual on/off switches for each distal end which are pressed when a desired distal end wants/needs to be actuated. In some embodiments, as mentioned above, the user can choose which distal end to actuate by pressing the necessary switches.

### Exemplary Motorized Release Tool

In some embodiments, in either a single or a multiple release tool configuration, the release tool is a motorized release tool. In some embodiments, the tool comprises an electric motor at the proximal end configured to actuate the rod in the same manner as the manual knob does. In some embodiments, the tool further comprises a button configured to be pressed by the user to activate the electrical motor of the tool. In some embodiments, additionally, the tool comprises a switch for choosing the direction of the rotation of the tool.

### Exemplary Methods

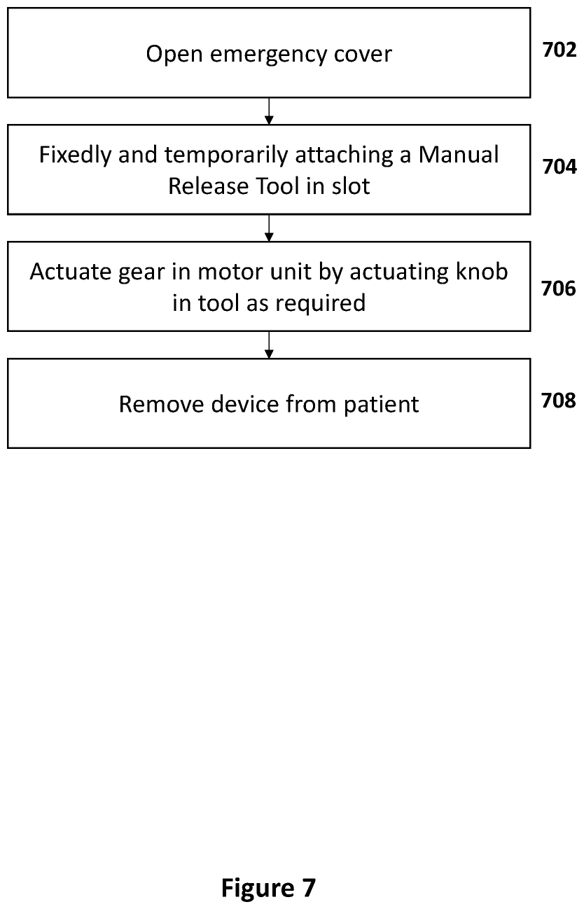

Referring now to FIG. 7, showing a flowchart of an exemplary method according to some embodiments of the invention. In some embodiments, the user inserts the robotic arms of the device inside the patient to perform the intervention. In some embodiments, the user then drives the robotic arm using an electronically controlled motor via a motor unit including at least one gear. In some embodiments, if and when there is a mechanical and/or electronic problem with the device and it is needed to be removed from the patient but the arms are folded and/or rotated and cannot be extracted, the user performs the following actions:
1. Removing the removable cover from the motor unit 702;
2. Fixedly and temporarily attaching the manual release tool to the motor unit 704;
3. Actuating/rotating the gear in the motor unit by actuating rotating the knob in the manual release tool; 706
4. Removing the device from the patient once the robotic arms are in a straight position. In some embodiments, this step is relevant during procedures alone. It should be noted that the tool can be used also while the device is not inside the patient and/or not during procedures.

### Exemplary Cradle

In some embodiments, the manual release tool is connected to the surgeon's console using a dedicated cradle. Referring now to FIG. 8a-b showing a schematic representation of a manual release tool in a dedicated cradle, according to some embodiments of the invention. In some embodiments, the manual release tool 400 is inserted into a dedicated cradle 802 and optionally secured with a latch 804. In some embodiments, the cradle can then be mounted/kept anywhere in the vicinity of the device and used when and if necessary. In some embodiments, the cradle 802 comprises one or more channels 806 configured to receive one or more protrusions 422 in the manual release tool 400. In some embodiments, the one or more channels 806 are used to assist in locking the manual release tool 400 in the cradle 802. For example, in order to lock in place the manual release tool 400 in the cradle 802, a user inserts the protrusions in the openings of the channel 806 (see arrow 808) and then it is moved towards the inside of the channel 806 (see arrow 810), which causes the distal end of the manual release tool 400 to interact with holder 812, which also assists in keeping the manual release tool 400 in place. After that, the user can close the latch 804.

As used herein with reference to quantity or value, the term “about” means “within ±20% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A manual release tool configured for manually rotating a gear of a motor unit of a motorized robotic system, comprising:
a. a body;
b. a knob rotatable around an axis of the body and located on a proximal side of said body;
c. a distal end of said body being narrower than said knob and comprising at least one first gear protruding axially from said distal end; said at least one first gear configured to operatively interact with at least one second gear of said motor unit of said motorized robotic system;
wherein force is transmitted by said knob to said at least one first gear using an axially oriented rod; said force being converted into rotation of said at least one first gear perpendicular to a longitudinal axis of said body and conveyed in a plane of said narrow distal end of said body by rotation of said at least one first gear in said plane.

2. The manual release tool according to claim 1, wherein said conveying is performed by a series of side by side gears operatively connected to said at least one first gear.

3. The manual release tool according to claim 1, further comprising at least one holder configured to hold said manual release tool in place on said robotic mechanism while said at least one first gear is being actuated.

4. The manual release tool according to claim 1, further comprising at least one marker on said tool for assisting a user to position said manual release tool on said motor unit.

5. The manual release tool according to claim 1, further comprising a motor in said body configured to allow motorized actuation of said at least one first gear.

6. The manual release tool according to claim 1, further comprising a cover-opening tool positioned at a distal end of said body and configured to assist a user to open a cover on said motor unit.

7. The manual release tool according to claim 1, wherein a width of said knob is from about 10 mm to about 60 mm.

8. The manual release tool according to claim 1, wherein a width of said distal end is from about 4 mm to about 10 mm.

9. The manual release tool according to claim 1, wherein said distal end is sized and shaped to be inserted in a narrow slot in said motor unit of said motorized robotic system.

10. The manual release tool according to claim 9, wherein a width of said slot is from about 6 mm to about 20 mm.

11. The manual release tool according to claim 1, wherein a ration between said at least one first gear and said at least one second gear is from about 1:1 to about 1:2.

12. The manual release tool according to claim 1, wherein said at least one first gear or said at least one second gear comprises between 10 and 40 teeth.

13. The manual release tool according to claim 1, wherein said manual release tool comprises more than one distal end configured to operatively interact with more than one gears of said motor unit of said motorized robotic system.

14. The manual release tool according to claim 1, further comprising a cradle configured for storing said manual release tool.

15. The manual release tool according to claim 14, further comprising at least one side protrusion on said body configured to interact with said cradle for holding said manual release tool in said cradle.

16. A robotic system comprising:
a. a motor unit comprising at least one gear;
b. at least one robotic arm operated by said motor unit;
c. an electrically controlled motor which powers said motor unit;
d. a manual release tool selectively and fixedly attachable to said motor unit and configured to engage and rotate said at least one gear.

17. The robotic system according to claim 16, wherein said manual release tool configured comprises:
a. a body;
b. a knob rotatable around an axis of the body and located on a proximal side of said body;
c. a distal end of said body being narrower than said knob and comprising at least one first gear protruding axially from said distal end; said at least one first gear configured to operatively interact with at least one second gear of said motor unit of said motorized robotic system; and
wherein force is transmitted by said knob to said at least one first gear using an axially oriented rod; said force being converted into rotation of said at least one first gear perpendicular to a longitudinal axis of said body and conveyed in a plane of said narrow distal end of said body by rotation of said at least one first gear in said plane.

18. The robotic system according to claim 17, wherein said conveying is performed by a series of side by side gears operatively connected to said at least one first gear.

19. The robotic system according to claim 17, further comprising at least one holder configured to hold said manual release tool in place on said robotic mechanism while said at least one first gear is being actuated.

20. The robotic system according to claim 17, further comprising at least one marker on said tool for assisting a user to position said manual release tool on said motor unit.

21. The robotic system according to claim 17, further comprising a motor in said body configured to allow motorized actuation of said at least one first gear.

22. The robotic system according to claim 17, further comprising a cover-opening tool positioned at a distal end of said body and configured to assist a user to open a cover on said motor unit.

23. The robotic system according to claim 17, wherein a width of said knob is from about 10 mm to about 60 mm.

24. The robotic system according to claim 17, wherein a width of said distal end is from about 4 mm to about 10 mm.

25. The robotic system according to claim 17, wherein said distal end is sized and shaped to be inserted in a narrow slot in said motor unit of said motorized robotic system.

26. The robotic system according to claim 25, wherein a width of said slot is from about 6 mm to about 20 mm.

27. The robotic system according to claim 17, wherein a ration between said at least one first gear and said at least one second gear is from about 1:1 to about 1:2.

28. The robotic system according to claim 17, wherein said at least one first gear or said at least one second gear comprises between 10 and 40 teeth.

29. The robotic system according to claim 17, wherein said manual release tool comprises more than one distal end configured to operatively interact with more than one gears of said motor unit of said motorized robotic system.

30. The robotic system according to claim 17, further comprising a cradle configured for storing said manual release tool.

31. The robotic system according to claim 30, further comprising at least one side protrusion on said body configured to interact with said cradle for holding said manual release tool in said cradle.

32. A manual release tool configured for manually rotating a gear of a motor unit of a motorized robotic system, comprising:
a. a body;
b. a knob rotatable around an axis of the body and located on a proximal side of said body;
c. a distal end of said body being narrower than said knob and comprising at least one first gear protruding axially from said distal end; said at least one first gear configured to operatively interact with at least one second gear of said motor unit of said motorized robotic system;
d. at least one motor located inside said body and operatively connected to said at least one first gear and configured for rotating said at least one first gear;
wherein force is transmitted by said motor to said at least one first gear using an axially oriented rod; said force being converted into rotation of said at least one first gear perpendicular to a longitudinal axis of said body and conveyed in a plane of said narrow distal end of said body by rotation of said at least one first gear in said plane.

33. The manual release tool according to claim 32, wherein said knob is configured to allow manual actuation of said at least one first gear when said at least one motor cannot be operated.

34. The manual release tool according to claim 32, further comprising circuitry configured to control said at least one motor.

35. The manual release tool according to claim 34, wherein said circuitry is operable by a user using one or more of a button, an external device connected via wireless connection and a GUI.

36. The manual release tool according to claim 32, wherein said conveying is performed by a series of side by side gears operatively connected to said at least one first gear.

37. The manual release tool according to claim 32, further comprising at least one holder configured to hold said manual release tool in place on said robotic mechanism while said at least one first gear is being actuated.

38. The manual release tool according to claim 32, further comprising at least one marker on said tool for assisting a user to position said manual release tool on said motor unit.

39. The manual release tool according to claim 32, further comprising a cover-opening tool positioned at a distal end of said body and configured to assist a user to open a cover on said motor unit.

40. The manual release tool according to claim 32, wherein a width of said knob is from about 10 mm to about 60 mm.

41. The manual release tool according to claim 32, wherein a width of said distal end is from about 4 mm to about 10 mm.

42. The manual release tool according to claim 32, wherein said distal end is sized and shaped to be inserted in a narrow slot in said motor unit of said motorized robotic system.

43. The manual release tool according to claim 42, wherein a width of said slot is from about 6 mm to about 20 mm.

44. The manual release tool according to claim 32, wherein a ration between said at least one first gear and said at least one second gear is from about 1:1 to about 1:2.

45. The manual release tool according to claim 32, wherein said at least one first gear or said at least one second gear comprises between 10 and 40 teeth.

46. The manual release tool according to claim 32, wherein said manual release tool comprises more than one distal end configured to operatively interact with more than one gears of said motor unit of said motorized robotic system.

47. The manual release tool according to claim 32, further comprising a cradle configured for storing said manual release tool.

48. The manual release tool according to claim 47, further comprising at least one side protrusion on said body configured to interact with said cradle for holding said manual release tool in said cradle.

49. A method of actuating a robotic arm, comprising:
a. driving said robotic arm using an electronically controlled motor via a motor unit including at least one first gear;
b. fixedly and temporarily attaching a manual release tool to said motor unit;
c. rotating said at least one first gear using said manual release tool.

50. The method according to claim 49, wherein said rotating comprises rotating at least one second gear located at a distal end of said manual release tool and configured to operatively engage said at least one first gear.

51. The method according to claim 49, wherein said rotating comprises manually rotating a knob located at a proximal end of said manual release tool.

52. The method according to claim 50, wherein said rotating comprises activating at least one motor located inside a body of said manual release tool and configured for rotating said at least one second gear.

53. The method according to claim 49, wherein said fixedly and temporarily attaching comprises attaching a holder located in said manual release tool to a dedicated location in said motor unit.
